(19) United States
(12) Patent Application Publication
(10) Pub. No.: US 2011/0142766 A1
(43) Pub. Date: Jun. 16, 2011

(54) Effervescent Multi-Vitamin Formulation and Methods of Use Thereof

(76) Inventor: Sal Rafanelli, Wayne, NJ (US)

(21) Appl. No.: 12/646,940

(22) Filed: Dec. 23, 2009

Related U.S. Application Data
(60) Provisional application No. 61/287,161, filed on Dec. 16, 2009.

(51) Int. Cl. A61K 9/00; A61P 3/02; A61P 27/02

(52) U.S. Cl. 424/43

(57) ABSTRACT

The present invention provides an effervescent dietary supplement formulation that may be beneficial to the management of symptoms related to ocular diseases. Also provided are methods of treating an ocular disease, such as macular degeneration, by administering the dietary supplement to a subject in need thereof.

### CROSS REFERENCE TO RELATED APPLICATION(S)

This application claims the benefit of priority under 35 U.S.C. §119(e) of U.S. Ser. No. 61/287,161, filed Dec. 16, 2009, the entire content of which is incorporated herein by reference.

### BACKGROUND OF THE INVENTION

1. Field of the Invention

The invention relates generally to a composition of a dietary supplement for treating ocular disorders.

2. Background Information

Age-related macular degeneration (AMD) and cataract are leading causes of visual impairment and blindness in the United States. Approximately 13 million Americans have some form of AMD, a majority of which may become blind from the disease if untreated. The National Eye Institute (NEI), one of the Federal government's National Institutes of Health, estimates that there are 1.5 million surgeries for cataract each year in the U.S. Based on many clinical studies, the frequency of both diseases increases dramatically after age 65.

Animal studies, observational epidemiologic studies, and a few small clinical trials had previously suggested that antioxidants and the trace elements zinc and selenium may be associated with the risk of AMD and cataract development. Also, a small, randomized clinical trial of zinc supplementation found a statistically significant reduction in vision loss in a group treated with zinc compared with a group treated with a placebo.

To help address the increasing public health concern regarding widespread use of high-dose vitamins and minerals for age-related macular degeneration, the NEI sponsored a major clinical study called The Age-Related Eye Disease Study (AREDS). AREDS scientists found that people at high risk for developing advanced AMD—those with intermediate AMD, and those with advanced AMD in one eye only—reduced their risk of developing advanced stages of AMD by about 25 percent when treated with a certain combination of antioxidants plus zinc. The combination of antioxidants plus zinc also reduced the risk of central vision loss by 19 percent in the same group. Participants at high risk for developing advanced AMD who were treated with zinc alone reduced their risk of developing advanced AMD by about 21 percent and their risk of vision loss by about 11 percent. Participants who were treated with antioxidants alone reduced their risk of developing advanced stages of AMD by about 17 percent and their risk of vision loss by about 10 percent.

The oral administration of medicaments, including vitamins, minerals, and antioxidants, to both pediatric and adult patient populations can often be a challenge. Particularly, patients are often reluctant to swallow pills, tablets, capsules, or other solid dosage medicament formulations, especially when the act of swallowing is problematic for that individual. For example, global hystericus and choking due to pharyngeal and esophageal motility problems, renders it painful to swallow and often results in aversion to swallowing the formulation. Patients may also be reluctant to ingest a medicament formulation due to its size, shape, and taste, psychological aversion to the act of ingestion, and/or personal choice not to swallow the formulation. However, patients under a medication regimen and/or in need of the therapeutic active ingredient in the formulation must self-administer, or be administered, the dose. Thus, there is a need to provide a better method of administering a therapeutically effective dose of vitamins, minerals, and antioxidants, for treating ocular diseases associated with deficiencies thereof.

### SUMMARY OF THE INVENTION

The present invention is based on the finding that certain vitamins, minerals, and antioxidants inhibit and/or ameliorate the symptoms associated with ocular disorders. Accordingly, in one aspect, the invention provides a dietary supplement formulation that includes an effervescing agent; vitamins, such as vitamin A, vitamin C, vitamin D, vitamin E, vitamin K, thiamin, riboflavin, niacin, vitamin B₆, folate, vitamin B₁₂, biotin, and pantothenic acid; minerals, such as calcium, phosphorus, iodine, magnesium, zinc, selenium, copper, manganese, chromium, molybdenum, chloride, sodium, potassium, nickel, silicon, boron, and vanadium; and antioxidants, such as lutein, zeaxanthin, and lycopene.

In another aspect, the present invention is based on the finding that certain vitamins, minerals, and antioxidants inhibit and/or ameliorate the symptoms associated with ocular disorders. Accordingly, the present invention provides a method of treating an ocular disease. The method includes administering to a subject in need thereof a therapeutically effective amount of the dietary supplement provided herein. In one embodiment, the dietary supplement is added to an aqueous vehicle, such as water, prior to administration to the subject. In another embodiment, the ocular disease is macular degeneration (including atrophic/dry and exudative/wet macular degeneration), diabetic retinopathy, choroidal neovascularization, and cataract. In another embodiment, the dietary supplement is administered to the subject daily. In another embodiment, the dietary supplement is administered to the subject once per day.

In another aspect, the present invention provides a method of treating macular degeneration in a subject. The method includes administering to a subject in need thereof a therapeutically effective amount of the dietary supplement provided herein. In one embodiment, the dietary supplement is added to an aqueous vehicle, such as water, prior to administration to the subject. In another embodiment, the dietary supplement is administered to the subject daily. In another embodiment, the dietary supplement is administered to the subject once per day.

### DETAILED DESCRIPTION OF THE INVENTION

The present invention is based on the finding that certain vitamins, minerals, and/or antioxidants slow the progression of various ocular diseases.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

The term “aqueous vehicle,” as used herein, is intended to refer to a medium or a carrier, such as a foodstuff, containing at least a minimal amount of water. Thus, the aqueous vehicle may be an oligohydrous vehicle containing a small amount of water, or it may be a vehicle having an abundance of water contained therein.

The term “foodstuff,” as used herein, is intended to refer to any safe, consumable liquid, semi-solid, or solid substance. Thus, a foodstuff would include any beverage and any food, which may be consumed by mammals of all classes and ages.

In one aspect, the invention provides a dietary supplement including one or more vitamins, one or more minerals, one or more antioxidants, and an effervescing agent, which facilitates delivery of the vitamins, minerals, and antioxidants contained in the dietary supplement to the subject in need of treatment. The dietary supplement is placed in an aqueous vehicle wherein the effervescing agent releases a one or more gases, which generally serve to penetrate and disperse the vitamins and minerals within the vehicle.

As used herein, the term “vitamin,” refers to any of a group of organic substances essential in small quantities to normal metabolism in a subject. As such, the term “vitamin” includes, without limitation, thiamin, riboflavin, niacin, nicotinic acid, pantothenic acid, pyridoxine, biotin, folic acid, vitamin B₆, vitamin B₁₂, lipoic acid, ascorbic acid (vitamin C), vitamin A, vitamin D, vitamin E and vitamin K and derivatives thereof. Also included within the term “vitamin” are the coenzymes thereof, as coenzymes are generally beneficial agents for the body. Coenzymes include thiamine pyrophosphates (TPP), flavin mononucleotides (FMM), flavin adenine dinucleotides (FAD), Nicotinamide adenine dinucleotides (AND), Nicotinamide adenine dinucleotide phosphate (NADP), Coenzyme-A (CoA) pyridoxal phosphate, biocytin, tetrahydrofolic acid, coenzyme B₁₂, lipoyllysine, 1,1-cis-retinal, and 1,2,5-dihydroxycholecalciferol. The term “vitamin” also includes choline, carnitine, and alpha, beta, and gamma carotenes. Thus, a vitamin may include, for example, substances that may or may not be required in the diet. Salts of vitamins are also suitable.

In one embodiment, the vitamins contained in the dietary supplement of the invention include vitamin A, vitamin C, vitamin D, vitamin E, vitamin K, thiamin, riboflavin, niacin, vitamin B₆, folate, vitamin B₁₂, biotin, and pantothenic acid. In another embodiment, the vitamin A is present as beta-carotene, the vitamin C is present as ascorbic acid, the vitamin D is present as cholecalciferol, the vitamin E is present as dl-alpha-tocopheryl acetate, the vitamin K is present as phytonadione, the thiamin is present as thiamin HCl, the niacin is present as niacinamide, the Vitamin B₆ is present as pyridoxine HCl, the folate is present as folic acid, the vitamin B₁₂ is present as cyanobalamin, the pantothenic acid is present as D-calcium pantothenate.

An effective amount of each vitamin contained in the dietary supplement of the invention is generally at least about 10% of the United States Recommended Daily Allowance (“RDA”) for a patient. For example, an effective amount of vitamin C would include an amount of vitamin C sufficient to provide 10% or more of the RDA. However, it should be understood that the amount of each vitamin component may be less than 10% of the RDA, may exceed 100% of the RDA, or may be present regardless of whether the U.S. issued an RDA therefor. Thus, in one embodiment, the vitamin A is present in an amount of about 500 IU to about 3500 IU, the vitamin C is present in an amount of about 500 mg to about 550 mg, the vitamin D is present in an amount of about 500 IU to about 1000 IU, the vitamin E is present in an amount of about 400 IU to about 1000 IU, the vitamin K is present in an amount of about 10 mcg to about 25 mcg, the thiamin is present in an amount of about 10 mg to about 12 mg, the riboflavin is present in an amount of about 8 mg to about 12 mg, the niacin is present in an amount of about 2 mg to about 10 mg, the vitamin B₆ is present in an amount of about 10 mg to about 20 mg, the folate is present in an amount of about 400 mcg to about 800 mcg, the vitamin B₁₂ is present in an amount of about 80 mcg to about 120 mcg, the biotin is present in an amount of about 6 mcg to about 30 mcg, and the pantothenic acid is present in an amount of about 10 mg to about 50 mg. In another embodiment, the vitamin A is present in an amount of about 3500 IU, the vitamin C is present in an amount of about 500 mg, the vitamin D is present in an amount of about 1000 IU, the vitamin E is present in an amount of about 400 IU, the vitamin K is present in an amount of about 10 mcg, the thiamin is present in an amount of about 10 mg, the riboflavin is present in an amount of about 10 mg, the niacin is present in an amount of about 20 mg, the vitamin B₆ is present in an amount of about 3 mg, the folate is present in an amount of about 800 mcg, the vitamin B₁₂ is present in an amount of about 100 mcg, the biotin is present in an amount of about 30 mcg, and the pantothenic acid is present in an amount of about 50 mg.

As used herein, the term “mineral” refers to inorganic substances that are generally required in the diet. Thus, suitable minerals include, but are not limited to, calcium, iron, zinc, selenium, copper, iodine, magnesium, manganese, phosphorus, chromium, molybdenum, chloride, sodium, potassium, nickel, silicon, boron, vanadium, salts thereof, chelates, and other compositional forms and combinations thereof.

In one embodiment, the minerals contained in the dietary supplement of the invention include calcium, phosphorus, iodine, magnesium, zinc, selenium, copper, manganese, chromium, molybdenum, chloride, sodium, potassium, nickel, silicon, boron, and vanadium. In another embodiment, the calcium is present as one or more of calcium carbonate and dicalcium phosphate, the magnesium is present as magnesium oxide, the zinc is present as zinc oxide, the selenium is present as sodium selenate, the copper is present as copper oxide, the manganese is present as manganese citrate, the chromium is present as chromium dinicotinate glycinate, the molybdenum is present as sodium molybdate, the chloride is present as potassium chloride, the sodium is present as sodium bicarbonate, the potassium is present as one or more of potassium carbonate, potassium chloride, and potassium bicarbonate, the nickel is present as nickel sulfate, the silicon is present as silicon dioxide, the vanadium is present as vanadium amino acid chelate, and the boron is present as boron chelate.

An effective amount of each mineral contained in the dietary supplement of the invention is generally at least about 10% of the United States Recommended Daily Allowance (“RDA”) for a patient. For example, an effective amount of calcium would include an amount of calcium sufficient to provide 10% or more of the RDA. However, it should be understood that the amount of each mineral component may be less than 10% of the RDA, may exceed 100% of the RDA, or may be present regardless of whether the U.S. issued an RDA therefor. Thus, in one embodiment, the calcium is present in an amount of about 200 mg to about 350 mg, the phosphorus is present in an amount of about 48 mg to about 100 mg, the iodine is present in an amount of about 85 mcg to about 150 mcg, the magnesium is present in an amount of about 85 mg to about 100 mg, the zinc is present in an amount of about 80 mg to about 90 mg, the selenium is present in an amount of about 20 mcg to about 40 mcg, the copper is present in an amount of about 2 mg to about 3.5 mg, the manganese is present in an amount of about 0.5 mg to about 2.5 mg, the chromium is present in an amount of about 100 mcg to about 250 mcg, the molybdenum is present in an amount of about 60 mcg to about 80 mcg, the chloride is present in an amount of about 60 mg to about 72 mg, the sodium is present in an amount of about 10 mg to about 15 mg, the potassium is present in an amount of about 300 mg to about 500 mg, the nickel is present in an amount of about 3 mcg to about 7 mcg, the silicon is present in an amount of about 30 mg to about 40 mg, the boron is present in an amount of about 100 mcg to about 150 mcg, and the vanadium is present in an amount of about 10 mcg to about 15 mcg. In another embodiment, the calcium is present in an amount of about 200 mg, the phosphorus is present in an amount of about 48 mg, the iodine is present in an amount of about 150 mcg, the magnesium is present in an amount of about 100 mg, the zinc is present in an amount of about 80 mg, the selenium is present in an amount of about 20 mcg, the copper is present in an amount of about 2 mg, the manganese is present in an amount of about 2 mg, the chromium is present in an amount of about 150 mcg, the molybdenum is present in an amount of about 75 mcg, the chloride is present in an amount of about 72 mg, the sodium is present in an amount of about 15 mg, and the potassium is present in an amount of about 500 mg.

As used herein, the term “antioxidant” refers to any of various substances that inhibit oxidation or reactions promoted by oxygen and peroxides and that include many held to protect the living body from the deleterious effects of free radicals. It should be understood that certain vitamins and/or minerals are known to be antioxidants, and while those components may be listed above, they are included within the term “antioxidant.” In one embodiment, the antioxidants contained in the dietary supplement of the invention include lutein, zeaxanthin, and lycopene.

An effective amount of each antioxidant contained in the dietary supplement of the invention is generally at least about 10% of the United States Recommended Daily Allowance (“RDA”) for a patient. For example, an effective amount of beta-carotene would include an amount of beta-carotene sufficient to provide 10% or more of the RDA. However, it should be understood that the amount of each antioxidant component may be less than 10% of the RDA, may exceed 100% of the RDA, or may be present regardless of whether the U.S. issued an RDA therefor. Thus, in one embodiment, the lutein is present in an amount of about 7 mg to about 10 mg, the zeaxanthin is present in an amount of about 2 mg to about 6 mg, and the lycopene is present in an amount of about 200 mcg to about 400 mcg. In another embodiment, the lutein is present in an amount of about 10 mg, the zeaxanthin is present in an amount of about 2 mg, and the lycopene is present in an amount of about 300 mcg.

As used herein, the term “effervescence” generally means the escape of a gas from a liquid or mixture (Hawley's Chemical Dictionary, pp. 432, 2001). Thus, the term “effervescent agent,” is intended to generally refer to a composition or mixture of components that evolve one or more gases, under proper conditions, such as upon contact with water.

In one embodiment, the effervescing agent is a mixture of compounds that evolve gas. These compounds should be capable of reacting upon exposure of one or both of the reactants to water, such as the water contained in aqueous fluids or other aqueous vehicles, to produce and/or evolve the gas. In one embodiment, the effervescing mixture includes at least one acidic component and at least one basic component. In this instance, the acidic and basic components react, upon exposure to water, with one another to produce at least one gas. For example, the reaction between a soluble acid, or source thereof, with an carbonate, or source thereof such as an alkaline metal carbonate, generally evolves CO₂ gas. More particularly, when such a gas-generating effervescent mixture is placed in a minimal amount of water, or water-containing vehicle such as saliva, CO₂ gas is generally produced and bubbles out of the water or aqueous vehicle.

The acidic component may be an acid or source thereof and should be safe for consumption. Suitable acids include, but are not limited to, food acids, acid anhydrides and acid salts. Exemplary food acids include, but are not limited to, citric acid, tartaric acid, malic acid, fumaric acid, adipic acid, and succinic acid. Anhydrides of the above-described acids may also be used because anhydrides generally degrade in the presence of water to generate the reactive acid. Exemplary suitable acid salts include, but are not limited to, sodium dihydrogen phosphate, disodium dihydrogen pyrophosphate, acid citrate salts and sodium acid sulfite. Acid salts generally disassociate in water, or in the water content of the aqueous vehicle, to provide the reactive acid species. The overall solubility of the acid, or source thereof, in water will vary and is appreciated by those of skill in the art. The effectiveness of the acid in generating the gas, and the amount of gas generated, is generally dependent on water solubility of the acid form in the dietary supplement.

Similarly, the basic component may be a carbonate or source thereof and should be safe for consumption. Suitable carbonate sources include, but are not limited to, dry solid carbonate, bicarbonate, sesquicarbonate, and sesquibicarbonate salts of metals, such as sodium, potassium, lithium, calcium, and magnesium. Examples of suitable carbonates include, without limitation, sodium carbonate, sodium bicarbonate, sodium sesquicarbonate, potassium carbonate, potassium bicarbonate, potassium sesquicarbonate, magnesium carbonate, sodium glycine carbonate, L-lysine carbonate, arginine carbonate, and amorphous calcium carbonate. Ammonium carbonate and ammonium bicarbonate are also suitable carbonates. In addition, any combination of the above sources of carbonate may be used as the basic component in the effervescing mixture.

It should be understood that the gas-generating effervescent component(s) are not limited to components reactive to form only carbon dioxide gas. Pharmaceutically safe reactants that evolve oxygen, nitrogen, helium, ethylene oxide, or other inert gases are also considered within the scope of the invention. For example, peroxides such as hydrogen peroxide, sodium peroxide and the like are capable of releasing useful oxygen gas. The combination of horse-radish with hydrogen peroxidase for example, or a vegetable peroxidase, is known to evolve oxygen gas. In addition, the gas-generating effervescent component(s) are not limited to mutually reactive components, such as the acidic and basic components described above, but may include safe, compounds, reactive with water to release a gas. Use of safe gas-generating effervescent component(s) and gases generated therefrom is particularly important in dietary supplements designed for oral administration.

The effectiveness of the effervescing agent to disperse the vitamins, minerals, and antioxidants of the dietary supplement is generally related to the degree of “pop” caused by the abrupt release of gas. As disclosed in U.S. Pat. No. 4,837,039, the disclosure of which is incorporated herein by reference in its entirety, the quantity and intensity of each “pop” is generally dependent upon the size of the bubble, the pressure of the gas contained in the bubble, the surface tension of the bubble, and the degree of solubility of the ingredients of the solid matrices in water or an aqueous vehicle. For example, the intensity of the release of gas depends upon the relation of the pressure of the occluded gas to resistance of the film of the bubble and on the diameter of the bubble trapping the gas.

The dietary supplement formulation may further include one or more additional adjuvants, which can be chosen from those known in the art. For example, adjuvants including flavors, sweeteners, colors, binders, diluents, filler, compaction agents, non-effervescent disintegrants, and the like, commonly referred to as excipients, may be included.

Suitable flavors for use in the dietary supplement formulation may be chosen from synthetic flavor oils and flavoring aromatics and/or natural oils, extracts from plants, leaves, flowers, fruits and so forth and combinations thereof. These may include cinnamon oil, oil of wintergreen, peppermint oils, clove oil, bay oil, anise oil, eucalyptus, thyme oil, cedar leave oil, oil of nutmeg, oil of sage, oil of bitter almonds and cassia oil. Also useful as flavors are vanilla, citrus oil, including lemon, orange, grape, lime and grapefruit, and fruit essences, including apple pear, peach, strawberry, raspberry, cherry, plum, pineapple, apricot and so forth. Flavors, which have been found to be particularly useful, include commercially available orange, grape, cherry and bubble gum flavors and mixtures thereof. The amount of flavoring may depend on a number of factors, including the organoleptic effect desired. Flavors may be present in an amount ranging from about 0.5% to about 3.0% by weight of the composition. Commonly accepted flavors include grape and cherry flavors, and citrus flavors such as orange. It is also appreciated that inclusion of flavoring agents can influence the final flavor of the vehicle, furthering compliance with ingestion of the dietary supplement.

Suitable sweeteners for use in the composition of the invention include, but are not limited to, carbohydrates, mono-saccharides, di-saccharides, poly-saccharides of simple sugars, and sugar derivatives. Exemplary sweeteners include, but are not limited to, high caloric sugars such as sucrose, lactose, glucose, d-glucose, I-glucose, maltose, dextrose, fructose, fructosan, gentiobiose, cellobiose, panose, malto-triose, malto-tetrose, arabinose, mannose, d-mannose, galactose, d-galactose, d-glyceraldehyde, amylose, allose, altose, talose, gulose, idose, ribose, erythrose, threose, lyxose, xylose, d-xylose, rhamnose, invert sugar, corn sugar, inositol, glycerol, glycogen, pectin, agar, sorbitol, mannitol and combinations thereof; low caloric sugars, such as sucralose, polyols, tagarose, trehalose, xylitol, dextrans, dextrins, dextrates, polysorbates, maltodextrin, xylitol, amylase, amylopectin, ribose, β-maltose, fucose, sialic acid (neuraminic acid), N-acetylgalactosamine, N-acetylglucosamine, sedoheptulose, ribulose, xylulose and combinations thereof; non-sugar sweeteners, such as acesulfane potassium, aspartame, neotame, saccharin, stevioside and combinations thereof.

Coloring agents may include titanium dioxide, and dyes suitable for food such as those known as F. D. & C. dyes and natural coloring agents such as grape skin extract, beet red powder, beta-carotene, annato, carmine, turmeric, paprika, etc. The amount of coloring used may range from about 0.1% to about 3.5% by weight of the final composition.

Examples of binders which can be used include acacia, tragacanth, gelatin, starch, cellulose materials such as methyl cellulose and sodium carboxy methyl cellulose, alginic acids and salts thereof, magnesium aluminum silicate, polyethylene glycol, guar gum, polysaccharide acids, bentonites, sugars, invert sugars and the like. Binders may be used in an amount up to about 60% by weight and advantageously from about 10% to about 40% by weight of the total composition.

Non-effervescent disintegrants include starches as corn starch, potato starch and modified starches thereof, sweeteners, clays, such as bentonite, micro-crystalline cellulose, alginates, gums such as agar, guar, locust bean, karaya, pecitin and tragacanth. Disintegrants may comprise up to about 20% by weight and advantageously between about 2% and about 10% by weight of the final composition. Notably, these binders and disintegrants may already be sufficiently present in other components of the formulation, such as in the effervescing mixture.

The individual components of the dietary supplement are formulated into a solid composition for placement in an aqueous vehicle. Suitable solid compositions include, without limitation, orally dispersable pills, chewable pills, buccal adhesive pills, tablets, capsules including hard or soft-shelled gelatin capsules, granular powder, troches, and dragees. These formulations may be prepared by techniques known in the art. For example, a pill may be manufactured by well-known pill manufacturing procedures.

Known granulation and wet-granulation methods for forming tablets may be utilized. Granulation generally includes any process of size enlargement whereby small particles are gathered together into larger, permanent aggregates to yield a free-flowing composition having a consistency suitable for tableting. Such granulated compositions may have consistency similar to that of dry sand. Granulation may be accomplished by agitation in mixing equipment or by compaction, extrusion or globulation. Granulation also includes, for example, a process where a liquid form of a material is rendered granular, or in a solid form, by combining it with a granular core material, such as a sugar particle. Such granular material may be produced, for example, by spray-drying the liquid onto the core particle. Thus, individual materials maybe granulated to lend themselves to tableting.

Lubricants are normally used in the manufacture of effervescent tablets. Without the use of an effective lubricant, tableting by use of high-speed equipment may be difficult. As used herein, the term “lubricant” refers to a material that can reduce the friction arising at the interface of the tablet and the die wall during compression and ejection thereof. Lubricants may also serve to prevent sticking to the punch and, to a lesser extent, the die wall as well. Lubricants, suitable for the effervescent formulation, may be used in an amount of up to 1.5% by weight, and advantageously between about 0.5% and about 1.0% by weight of the total composition.

Extrinsic or intrinsic lubricants may be incorporated in the material to be tableted. A lubricant that is directly applied to the tableting tool surface in the form of a film, as by spraying onto the die cavity and/or punch surfaces, is known as an extrinsic lubricant. Although extrinsic lubricants can provide effective lubrication, their use requires complex application equipment and methods which add cost and reduce productivity. Magnesium, calcium and zinc salts of stearic acid have long been regarded as the most efficient intrinsic lubricants in common use. Concentrations of 1% or less by weight are usually effective.

Other traditional intrinsic lubricants include hydrogenated and partially hydrogenated vegetable oils, animal fats, polyethyleneglycol, polyoxyethylene monostearate, talc, light mineral oils, sodium benzoate, sodium lauryl sulphate, magnesium oxide and the like. See Leal, et al., U.S. Pat. No. 3,042,531, the disclosure of which is incorporated herein by reference in its entirety.

The dietary supplement may be formulated to optimize exposure of the effervescing agent to the water content of the aqueous vehicle. For example, the formulation may contain a plurality of layers including an outermost layer and a core. Any of the components, including the effervescing agents, vitamins, minerals, and antioxidants, may be included in the outermost layer or distributed as desired between the outermost layer and the core. Thus, bi-layered or multi-layered tablet or pill formulations are contemplated herein.

In yet another embodiment, the dietary supplement is varied in shape. For example, while conventional oval shapes of a tablet or round shapes of a pill exist, the formulation may be provided in a non-traditional shape so as to increase the surface area of the formulation that is exposed to the vehicle. Particularly, for oligohydrous vehicles having minimal water content, exposing a maximum surface area of the formulation will enhance the rate of effervescence, thereby promoting the rate of distribution of the vitamins, minerals, and antioxidants into the vehicle. Generally, patients do not prefer to wait for a lengthy period of time before ingesting the vehicle. Therefore, in one embodiment of the invention, the solid formulation, such as a tablet or pill, has a biconcave shape to increase the surface area for contact with the vehicle. Such a shape may also comprise multiple layers, as previously discussed herein, wherein one or more layers contain one or more of the components of the dietary supplement. Optimal exposure of these components generally minimizes the time required to disperse the vitamins, minerals, and antioxidants into the vehicle by the effervescence of gas.

As the effervescing agents are water activated, the dietary supplement formulation should be kept in a generally dry environment with little or no moisture available to be absorbed. Accordingly, the components themselves should be generally anhydrous or in a stable hydrated form as exposure to water may prematurely disintegrate the effervescent formulation. Alternatively, a dessicant such, as calcium carbonate, may be included to keep the formulation dry.

The effervescent formulation may be orally administered to the patient in a variety of ways. In one way, it is initially placed in an aqueous vehicle, where it may be further stirred and/or agitated to commence effervescence of gases within the vehicle. Vehicles containing as little as about 0.1 ml of total water content are generally suitable to commence effervescence of gas(es) from the formulation. The effervescing gases promote penetration and distribution of the vitamins, minerals, and antioxidants into the vehicle. In one embodiment, the dietary supplement formulation is placed into a vehicle containing up to about 5 ml of water. In another embodiment, the dietary supplement formulation is placed into a vehicle containing up to about 6 ounces of water. In another embodiment, the effervescent formulation is placed in a vehicle containing between about 5 ml and about 15 ml of water. In yet another embodiment, the effervescent formulation is placed in a vehicle containing at least about 15 ml of water.

Another way of administering the formulation is by having the patient ingest the formulation directly. In such a case, the patient's saliva or other oral fluid acts as the vehicle in which the effervescence occurs. In the fluids in the patient's mouth, the formulation generally begins to disintegrate, commencing the production and/or evolution of gas. Thus, the amount of effervescing agent in the formulation should be effective to provide an effervescent sensation in the mouth of the patient. In other words, the patient should be able to perceive a distinct sensation of “fizzing” or bubbling and “popping” as the formulation disintegrates in the mouth. The “fizzing” sensation substantially enhances the organoleptic effects of the formulation. A “positive” organoleptic sensation is one which is pleasant and/or enjoyable and which can be perceived readily by a normal human being.

In either manner of administration, the effervescent formulation, should contain the effervescent components in amounts effective to assist the rapid and complete disintegration of the composition into the aqueous vehicle or in the mouth of the patient. By “rapid,” it is understood that the formulation should disintegrate in water, in an aqueous vehicle, or even in a patient's mouth in less than about 10 minutes, and desirably between about 30 seconds and about 7 minutes. In one embodiment of the invention, the formulation is a tablet which dissolves in the vehicle or mouth in between about 30 seconds and about 5 minutes. In another embodiment, it dissolves and disperses in less than about 30 seconds. Disintegration time can generally be measured by observing the disintegration time of the tablet in water at about 37° C. The tablet is immersed in the vehicle without forcible agitation. The disintegration time is the time from immersion for substantially complete dispersion of the tablet as determined by visual observation. This method for measuring disintegration times is only one of the many methods for such purpose, as known by those skilled in this art.

In another aspect, the invention provides methods for ameliorating/treating ocular diseases associated with a vitamin, mineral, and/or antioxidant deficiency, such as macular degeneration or cataract, in a subject. Most of the aging eye diseases progress slowly and a multitude of factors (genetic and environmental) affect their development and progression, so that it becomes very difficult to isolate the influence of a specific vitamin or mineral on this process. It therefore is reasonable to assume that strengthening of the eye defenses by increasing the intake of certain vitamins, minerals, and antioxidants would be helpful in preventing one or more chronic ocular diseases. Such ocular diseases include, but are not limited to, age-related macular degeneration (AMD), ocular histoplasmosis syndrome, pathologic myopia, diabetic retinopathy, angioid streaks, idiopathic disorders, and choroiditis, choroidal rupture, and cataract.

Age-related macular degeneration (AMD) is a collection of clinically recognizable ocular findings that can lead to blindness. The findings include drusen, retinal pigment epithelial (RPE) disturbance-including pigment clumping and/or dropout, RPE detachment, geographic atrophy, subretinal neovascularization and disciform scar. Not all these manifestations are needed for AMD to be considered present. Deposits under the retina called drusen are a common feature of macular degeneration. Drusen alone usually do not cause vision loss, but when they increase in size or number, this generally indicates an increased risk of developing advanced AMD. As such, macular degeneration can generally be understood to include deterioration or breakdown of the macula. The macula is a small area in the retina at the back of the eye that provides the ability to see fine details clearly. When the macula does not function correctly, central vision can be affected by blurriness, dark areas or distortion. As used herein, the terms “Dry” or “Dry Macular Degeneration” or “Atrophic Macular Degeneration” refer to macular degeneration caused by aging and thinning of the tissues of the macula, which typically results in gradual visual loss. As used herein, the terms “Wet” or “Wet Macular Degeneration” or “Exudative Macular Degeneration” refer to macular degeneration caused by abnormal blood vessels form underneath the retina at the back of the eye. These new blood vessels leak fluid or blood and blur central vision, which typically results in rapid and severe vision loss.

Accordingly, in one embodiment, the method for treating macular degeneration provided herein includes administering to a subject in need thereof a therapeutically effective amount of the dietary supplement described herein. As used herein, the term “ameliorating” or “treating” means that the clinical signs and/or the symptoms associated with an ocular disorder (e.g., macular degeneration) are lessened as a result of the actions performed. The signs or symptoms to be monitored will be characteristic of the ocular disorder (e.g., macular degeneration) and will be well known to the skilled clinician, as will the methods for monitoring the signs and conditions. For example, subjects having macular degeneration may experience fuzzy or blurry vision, an empty or dark area in the center of vision, the appearance of straight lines, such as sides of buildings, telephone poles, or sentences on a page, as curved or wavy, and/or a dimming of vision when reading. Also included in the definition of “ameliorating” or “treating” is the lessening of symptoms associated with the ocular disorders in subjects not yet diagnosed as having the specific disorders. As such, the methods may be used as a means for prophylactic therapy for a subject at risk of having a specific ocular disorder.

In one embodiment, the signs and symptoms associated with an ocular disorder (e.g., macular degeneration) may be monitored by assessment via Optical Coherence Tomography (OCT). OCT is a non-invasive, fast, non-contact imaging technique which readily displays intra-retinal, subretinal and sub-RPE fluid. OCT relies upon differential reflections of light to produce 2-dimensional cross-sections of the retina. OCT images are obtained rapidly and have a spatial resolution of approximately 8 mcm. OCT is especially useful for calculating retinal thickness. In another embodiment, the signs and symptoms associated with an ocular disorder (e.g., macular degeneration) may be monitored by assessment via a visual acuity (VA) test. The visual acuity test is used to determine the smallest letters a person can read on a standardized chart or card held 14-20 feet away. This test is done on each eye, one at a time. If necessary, it is then repeated while the subject wears glasses or contacts.

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of the term “subject.”

As used herein, the term “therapeutically effective amount” or “effective amount” means the amount of a compound or pharmaceutical composition that will elicit the biological or medical response of a tissue, system, animal or human that is being sought by the researcher, veterinarian, medical doctor or other clinician. The terms “administration” or “administering” are defined to include the act of providing a compound or pharmaceutical composition of the invention to a subject in need of treatment. While the dietary supplement of the invention is disclosed for oral administration, it should be understood that the composition may be administered by any means determined by the researcher, veterinarian, medical doctor or other clinician for treating the subject. Exemplary modes of administration include, but are not limited to, intravenously, intra-arterially, subcutaneously, intraperitoneally, intramuscularly, or orally. Thus, in one embodiment, the dietary supplement is orally ingested.

The total amount of the dietary supplement to be administered in practicing a method of the invention can be administered to a subject as a single dose or can be administered using a fractionated treatment protocol, in which multiple doses are administered over a prolonged period of time (e.g., once daily, twice daily, etc.). As such, in one embodiment, the dietary supplement is administered to the subject daily. In another embodiment, the dietary supplement is administered to the subject once per day.

In certain embodiments, the dietary supplement of the invention may further be administered in combination with an antiinflammatory, antimicrobial, antihistamine, chemotherapeutic agent, antiangiogenic agent, immunomodulator, therapeutic antibody or a protein kinase inhibitor, e.g., a tyrosine kinase inhibitor, to a subject in need of such treatment. Other agents that may be administered in combination with invention compounds include protein therapeutic agents such as cytokines, immunomodulatory agents and antibodies. While not wanting to be limiting, antimicrobial agents include antivirals, antibiotics, anti-fungals and anti-parasitics. When other therapeutic agents are contemplated for use in combination with the dietary supplement of the present invention, they may be used for example in amounts as noted in the Physician Desk Reference (PDR) or as otherwise determined by one having ordinary skill in the art.

The following examples are provided to further illustrate the advantages and features of the present invention, but are not intended to limit the scope of the invention. While they are typical of those that might be used, other procedures, methodologies, or techniques known to those skilled in the art may alternatively be used.

### EXAMPLE I

### Effervescent Dietary Supplement Formulation

One packet (8.0 g) of the dietary supplement is added to 6 ounces of water. The dietary supplement contains the following:

Vitamin A (as beta-carotene): 3500 IU

Vitamin C (as ascorbic acid): 500 mg

Vitamin D (as cholecalciferol): 100 IU

Vitamin E (as dl-alpha-tocopheryl acetate): 400 IU

Vitamin K (as phytonadione): 10 mcg

Thiamin (as thiamin HCl): 10 mg

Riboflavin: 10 mg

Niacin (as niacinamide): 20 mg

Vitamin B₆ (as pyridoxine HCl): 3 mg

Folate (as folic acid): 800 mcg

Vitamin B₁₂ (as cyanocobalamin): 100 mcg

Biotin: 30 mcg

Pantothenic Acid (as D-calcium pantothenate): 500 mg

Calcium (as calcium carbonate and dicalcium phosphate): 200 mg

Phosphorus (as dicalcium phosphate): 48 mg

Iodine (from kelp): 150 mcg

Magnesium (magnesium oxide): 100 mg

Zinc (as zinc oxide): 80 mg

Selenium (as sodium selenate): 20 mcg

Copper (as copper oxide): 2 mg

Manganese (as manganese citrate): 2 mg

Chromium (as chromium dinicotinate glycinate): 150 mcg

Molybdenum (as sodium molybdate): 75 mcg

Chloride (as potassium chloride): 72 mg

Sodium (as sodium bicarbonate): 15 mg

Potassium (as potassium carbonate, potassium chloride, and potassium bicarbonate): 500 mg

Lutein: 10 mg

Zeaxanthin: 2 mg

Nickel (as nickel sulfate): 5 mcg

Silicon (as silicon dioxide): 35 mg

Vanadium (as vanadium amino acid chelate): 10 mcg

Boron (as boron chelate): 150 mcg

Lycopene: 300 mcg

Other ingredients: citric acid, maltodextrin, potassium carbonate, potassium bicarbonate, natural flavors, acesulfame potassium, sodium bicarbonate and sucralose.

Although the invention has been described with reference to the above example, it will be understood that modifications and variations are encompassed within the spirit and scope of the invention. Accordingly, the invention is limited only by the following claims.

What is claimed is:

1. A dietary supplement comprising:
(a) an effervescing agent;
(b) vitamins, wherein the vitamins comprise: vitamin A, vitamin C, vitamin D, vitamin E, vitamin K, thiamin, riboflavin, niacin, vitamin B₆, folate, vitamin B₁₂, biotin, and pantothenic acid;
(c) minerals, wherein the minerals comprise: calcium, phosphorus, iodine, magnesium, zinc, selenium, copper, manganese, chromium, molybdenum, chloride, sodium, potassium, nickel, silicon, boron, and vanadium; and
(d) antioxidants, wherein the antioxidants comprise: lutein, zeaxanthin, and lycopene.

2. The supplement of claim 1, wherein the effervescing agent is a mixture of (a) at least one acid selected from the group consisting of citric acid, tartaric acid, malic acid, fumaric acid, adipic acid, succinic acid, acid anhydrides of the acids, and (b) at least one carbonate source selected from the group consisting of carbonate salts, bicarbonate salts, and sesequicarbonate salts.

3. The supplement of claim 1, wherein the vitamin A is present as beta-carotene, the vitamin C is present as ascorbic acid, the vitamin D is present as cholecalciferol, the vitamin E is present as dl-alpha-tocopheryl acetate, the vitamin K is present as phytonadione, the thiamin is present as thiamin HCl, the niacin is present as niacinamide, the Vitamin B₆ is present as pyridoxine HCl, the folate is present as folic acid, the vitamin B₁₂ is present as cyanobalamin, the pantothenic acid is present as D-calcium pantothenate, the calcium is present as one or more of calcium carbonate and dicalcium phosphate, the magnesium is present as magnesium oxide, the zinc is present as zinc oxide, the selenium is present as sodium selenate, the copper is present as copper oxide, the manganese is present as manganese citrate, the chromium is present as chromium dinicotinate glycinate, the molybdenum is present as sodium molybdate, the chloride is present as potassium chloride, the sodium is present as sodium bicarbonate, the potassium is present as one or more of potassium carbonate, potassium chloride, and potassium bicarbonate, the nickel is present as nickel sulfate, the silicon is present as silicon dioxide, the vanadium is present as vanadium amino acid chelate, and the boron is present as boron chelate.

4. The supplement of claim 1, wherein the vitamin A is present in an amount of about 500 IU to 3500 IU, the vitamin C is present in an amount of about 500 mg to 550 mg, the vitamin D is present in an amount of about 500 IU to 1000 IU, the vitamin E is present in an amount of about 400 IU to 1000 IU, the vitamin K is present in an amount of about 10 mcg to 25 mcg, the thiamin is present in an amount of about 10 mg to 12 mg, the riboflavin is present in an amount of about 8 mg to 12 mg, the niacin is present in an amount of about 2 mg to 10 mg, the vitamin B₆ is present in an amount of about 10 mg to 20 mg, the folate is present in an amount of about 400 mcg to 800 mcg, the vitamin B₁₂ is present in an amount of about 80 mcg to 120 mcg, the biotin is present in an amount of about 6 mcg to 30 mcg, and the pantothenic acid is present in an amount of about 10 mg to 50 mg.

5. The supplement of claim 1, wherein the calcium is present in an amount of about 200 mg to 350 mg, the phosphorus is present in an amount of about 48 mg to 100 mg, the iodine is present in an amount of about 85 mcg to 150 mcg, the magnesium is present in an amount of about 85 mg to 100 mg, the zinc is present in an amount of about 80 mg to 90 mg, the selenium is present in an amount of about 20 mcg to 40 mcg, the copper is present in an amount of about 2 mg to 3.5 mg, the manganese is present in an amount of about 0.5 mg to 2.5 mg, the chromium is present in an amount of about 100 mcg to 250 mcg, the molybdenum is present in an amount of about 60 mcg to 80 mcg, the chloride is present in an amount of about 60 mg to 72 mg, the sodium is present in an amount of about 10 mg to 15 mg, the potassium is present in an amount of about 300 mg to 500 mg, the nickel is present in an amount of about 3 mcg to 7 mcg, the silicon is present in an amount of about 30 mg to 40 mg, the boron is present in an amount of about 100 mcg to 150 mcg, and the vanadium is present in an amount of about 10 mcg to 15 mcg.

6. The supplement of claim 1, wherein the lutein is present in an amount of about 7 mg to 10 mg, the zeaxanthin is present in an amount of about 2 mg to 6 mg, and the lycopene is present in an amount of about 200 mcg to 400 mcg.

7. The supplement of claim 4, wherein the vitamin A is present in an amount of about 3500 IU, the vitamin C is present in an amount of about 500 mg, the vitamin D is present in an amount of about 1000 IU, the vitamin E is present in an amount of about 400 IU, the vitamin K is present in an amount of about 10 mcg, the thiamin is present in an amount of about 10 mg, the riboflavin is present in an amount of about 10 mg, the niacin is present in an amount of about 20 mg, the vitamin B₆ is present in an amount of about 3 mg, the folate is present in an amount of about 800 mcg, the vitamin B₁₂ is present in an amount of about 100 mcg, the biotin is present in an amount of about 30 mcg, and the pantothenic acid is present in an amount of about 50 mg.

8. The supplement of claim 5, wherein the calcium is present in an amount of about 200 mg, the phosphorus is present in an amount of about 48 mg, the iodine is present in an amount of about 150 mcg, the magnesium is present in an amount of about 100 mg, the zinc is present in an amount of about 80 mg, the selenium is present in an amount of about 20 mcg, the copper is present in an amount of about 2 mg, the manganese is present in an amount of about 2 mg, the chromium is present in an amount of about 150 mcg, the molybdenum is present in an amount of about 75 mcg, the chloride is present in an amount of about 72 mg, the sodium is present in an amount of about 15 mg, and the potassium is present in an amount of about 500 mg.

9. The supplement of claim 6, wherein the lutein is present in an amount of about 10 mg, the zeaxanthin is present in an amount of about 2 mg, and the lycopene is present in an amount of about 300 mcg.

10. A dietary supplement comprising:
(a) an effervescing agent;
(b) vitamins, wherein the vitamins comprise: about 3500 IU of vitamin A, about 500 mg of vitamin C, about 100 IU of vitamin D, about 400 IU of vitamin E, about 10 mcg of vitamin K, about 10 mg of thiamin, about 10 mg of riboflavin, about 20 mg of niacin, about 3 mg of vitamin B₆, about 800 mcg of folate, about 100 mcg of vitamin B₁₂, about 30 mcg of biotin, and about 500 mg of pantothenic acid;
(c) minerals, wherein the minerals comprise: about 200 mg of calcium, about 48 mg of phosphorus, about 150 mcg of iodine, about 100 mg of magnesium, about 80 mg of zinc, about 20 mcg of selenium, about 2 mg of copper, about 2 mg of manganese, about 150 mcg of chromium, about 75 mcg of molybdenum, about 72 mg of chloride, about 15 mg of sodium, about 500 mg of potassium, about 5 mcg of nickel, about 35 mg of silicon, about 150 mcg of boron, and about 10 mcg of vanadium; and
(d) antioxidants, wherein the antioxidants comprise: about 10 mg of lutein, about 2 mg of zeaxanthin, and about 300 mcg of lycopene.

11. A method of treating an ocular disease comprising administering to a subject in need thereof a therapeutically effective amount of the dietary supplement of claim 1.

12. The method of claim 11, wherein the supplement is added to water prior to administering the subject.

13. The method of claim 11, wherein the ocular disease is selected from the group consisting of macular degeneration, diabetic retinopathy, and cataract.

14. The method of claim 13, wherein the ocular disease is dry macular degeneration.

15. A method of treating macular degeneration comprising administering to a subject in need thereof a therapeutically effective amount of a dietary supplement, wherein the supplement comprises:
(a) an effervescing agent;
(b) vitamins, wherein the vitamins comprise: vitamin A, vitamin C, vitamin D, vitamin E, vitamin K, thiamin, riboflavin, niacin, vitamin B₆, folate, vitamin B₁₂, biotin, and pantothenic acid;
(c) minerals, wherein the minerals comprise: calcium, phosphorus, iodine, magnesium, zinc, selenium, copper, manganese, chromium, molybdenum, chloride, sodium, potassium, nickel, silicon, boron, and vanadium; and
(d) antioxidants, wherein the antioxidants comprise: lutein, zeaxanthin, and lycopene.

16. The method of claim 15, wherein the supplement is added to water prior to administering the subject.

17. The method of claim 15, wherein the vitamin A is present in an amount of about 3500 IU, the vitamin C is present in an amount of about 500 mg, the vitamin D is present in an amount of about 1000 IU, the vitamin E is present in an amount of about 400 IU, the vitamin K is present in an amount of about 10 mcg, the thiamin is present in an amount of about 10 mg, the riboflavin is present in an amount of about 10 mg, the niacin is present in an amount of about 20 mg, the vitamin B₆ is present in an amount of about 3 mg, the folate is present in an amount of about 800 mcg, the vitamin B₁₂ is present in an amount of about 100 mcg, the biotin is present in an amount of about 30 mcg, and the pantothenic acid is present in an amount of about 50 mg.

18. The method of claim 15, wherein the calcium is present in an amount of about 200 mg, the phosphorus is present in an amount of about 48 mg, the iodine is present in an amount of about 150 mcg, the magnesium is present in an amount of about 100 mg, the zinc is present in an amount of about 80 mg, the selenium is present in an amount of about 20 mcg, the copper is present in an amount of about 2 mg, the manganese is present in an amount of about 2 mg, the chromium is present in an amount of about 150 mcg, the molybdenum is present in an amount of about 75 mcg, the chloride is present in an amount of about 72 mg, the sodium is present in an amount of about 15 mg, and the potassium is present in an amount of about 500 mg.

19. The method of claim 15, wherein the lutein is present in an amount of about 10 mg, the zeaxanthin is present in an amount of about 2 mg, and the lycopene is present in an amount of about 300 mcg.

20. The method of claim 15, wherein the macular degeneration is dry macular degeneration.
